(19) United States
(12) Patent Application Publication
(10) Pub. No.: US 2018/0327805 A1
(43) Pub. Date: Nov. 15, 2018

(54) SYSTEM, METHOD AND COMPUTER PROGRAM PRODUCT FOR THE ORGANISM-SPECIFIC DIAGNOSIS OF SEPTICEMIA IN INFANTS

(71) Applicant: University of Virginia Patent Foundation d/b/a University of Virginia Licensing & Ventures Group, Charlottesville, VA (US)

(72) Inventors: Karen D. Fairchild, Charlottesville, VA (US); Jeffrey Saucerman, Crozet, VA (US); Randall J. Moorman, Keswick, VA (US); Douglas E. Lake, Charlottesville, VA (US)

(73) Assignee: University of Virginia Patent Foundation d/b/a University of Virginia Licensing & Ventures Group, Charlottesville, VA (US)

(21) Appl. No.: 15/877,051

(22) Filed: Jan. 22, 2018

Related U.S. Application Data
(63) Continuation of application No. 13/695,243, filed on Mar. 11, 2013, now abandoned.
(60) Provisional application No. 61/329,587, filed on Apr. 30, 2010.
(60) Provisional application No. 61/330,679, filed on May 3, 2010.

(51) Int. Cl. C12Q 1/04; G01N 33/68

(52) U.S. Cl. CPC C12Q 1/04; G01N 2800/38; G01N 2800/26; G01N 2333/5421; G01N 2333/5412; G01N 2333/535; G01N 2333/525; G01N 33/6869; G01N 33/6863

(57) ABSTRACT

A method, system, and computer program product for producing an organism specific diagnosis of septicemia in infants is disclosed. The method involves measuring the levels of one or more biomarkers against predefined threshold values and interpreting these levels to arrive at the diagnosis. Other techniques may introduce a preliminary step of identifying higher risk subjects, as well as the integration of such methods into the final diagnostic methodology. One aspect of a technique of this method may involve measuring one more cytokines to detect specific classes of infective organisms, such as Gram-negative bacteria.

### CROSS-REFERENCE TO RELATED APPLICATIONS

The present application claims priority from U.S. Provisional Application Ser. No. 61/329,587, filed Apr. 3, 2010, entitled “Method, System and Computer Program Product for Cytokines as Diagnostic Markers for Prediction of Neonatal Sepsis,” and U.S. Provisional Application Ser. No. 61/330,679, filed May 3, 2010, entitled “Method, System and Computer Program Product for Cytokines as Diagnostic Markers for Prediction of Neonatal Sepsis;” the disclosures of which are hereby incorporated by reference herein in their entirety.

### FIELD OF THE INVENTION

The present invention relates to the field of infant septicemia. More specifically, the present invention relates to the field of organism-specific diagnosis systems and methodology.

### BACKGROUND OF THE INVENTION

Infants in the Neonatal Intensive Care Unit (NICU) are highly susceptible to late-onset sepsis, with rates as high as 25% among preterm very low birth weight infants, leading to 45% of late deaths as well as more hospital days, mechanical ventilation, and antibiotic use. Furthermore, even those who survive are at increased risk for neurodevelopmental impairment. Diagnosis is difficult because the clinical signs are subtle and nonspecific, and lab tests including “gold standard” blood cultures are not very reliable. Presently, the standard paradigm for diagnosing and treating late-onset sepsis is to perform a blood culture and initiate empiric two-antibiotic therapy after an infant displays clinical signs and symptoms possibly attributable to sepsis. Unfortunately, with this approach, the mortality rate is high, particularly in cases of Gram-negative septicemia.

Thus, there is a need for diagnostic systems and techniques that allow for earlier and more accurate diagnosis of neonatal septicemia in order to substantially improve outcomes. Furthermore, there is a need for the ability to identify the likely causative organism so that antibiotic therapy can be tailored accordingly. Such diagnostic capabilities would also allow patients to avoid unnecessary antibiotic therapy.

Abnormal heart rate characteristics (HRC) have been identified as a novel physiomarker of neonatal sepsis and often occur prior to clinical deterioration. See Applicant's U.S. Pat. No. 7,774,050 B2, entitled “Method and Apparatus for the Early Diagnosis of Subacute, Potentially Catastrophic Illness.” Bacteremia can trigger a systemic inflammatory response with release of cytokines and subsequent physiologic changes in multiple organs including the heart. Two such changes identified in septic neonates are decreased beat-to-beat variability and repetitive transient decelerations in heart rate, similar to the changes seen in fetuses in the setting of asphyxia or chorioamnionitis. These abnormal heart rate characteristics are not apparent to clinicians using conventional cardiorespiratory monitoring, prompting development of a monitor that detects heart rate characteristics predictive of impending clinical deterioration. Through analysis of electrocardiogram data from hundreds of preterm infants, an HRC index was derived which incorporates decreased variability and decelerations to calculate a score, the fold increase in risk that a patient will be diagnosed with sepsis in the next 24 hours.

However, while sepsis has been identified as a major cause of decreased heart rate variability and transient decelerations in NICU patients, other conditions can also cause a rise in the HRC index. Other, more sepsis-specific tests are urgently needed.

Accordingly, an aspect of an embodiment of the present invention provides for, among other things, the use of a biomarker test for sepsis at the time of a rise in the HRC index that can aid clinicians in distinguishing patients with sepsis from those with non-septic conditions, and allow for the identification of the specific infective organism.

### SUMMARY OF THE INVENTION

An aspect of an embodiment proposes using, among other things, cytokines as a promising biomarker since some of them rise very early in the course of bacteremia.

An aspect of an embodiment provides, among other things, early identification of patients infected with Gram-negative organisms, through cytokine screening at the time of blood culture, thereby providing for a more timely initiation of broad-spectrum antibiotic combinations to more rapidly clear these highly virulent pathogens from the bloodstream, and might also serve to target patients for adjunct therapies to combat the detrimental effects of cytokine overproduction.

In addition to early diagnosis of septicemia and the identification of specific classes of infective organisms, another aspect of an embodiment of the present invention biomarker screening is, but not limited thereto, to provide the ability to rule out sepsis in patients with non-specific signs and symptoms.

Empiric antibiotic therapy for “sepsis rule-outs” is exceedingly common in NICU patients and consequently there is increasing evidence of adverse effects of antibiotic overuse. Accordingly, an aspect of an embodiment of the present invention will, at minimum, alleviate or mitigate the complications and problems associated with this phenomenon.

An aspect of an embodiment of the present invention provides, among other things, a method of determining the presence of a specific class of infective organism and/or blood culture result in an infant. The method may comprise: measuring the levels of one or more biochemical substances in one or more samples; assessing levels of the one or more biochemical substances against a target value; and interpreting the assessment to provide the determination of the presence of a specific class of infective organism or blood culture result in the infant.

An aspect of an embodiment of the present invention provides, among other things, a system for determining the presence of a specific class of infective organism and/or blood culture result in infants. The system may comprise: a sampling device for measuring the levels of one or more biochemical substances in one or more samples; one or more computer processing devices configured for assessing levels of the one or more biochemical substances against a target value; and interpreting the assessment to provide the determination of the presence of a specific class of infective organism or blood culture result in the infant.

An aspect of an embodiment of the present invention provides, among other things, a computer program product comprising a computer useable medium having a computer program logic for enabling at least one processor in a computer system determining the presence of a specific class of infective organism and/or blood culture result in an infant. The computer logic comprising (or the program is configured to, when executed by the processor, causes a system to operate at least by): measuring the levels of one or more biochemical substances in a sample; identifying and counting the number of the biochemical substances whose levels are above a threshold value; and interpreting the measures of the one or more circulating substances to provide the determination of the presence of a specific class of infective organism or blood culture result in the infant.

A method, system, and computer program product for producing an organism-specific diagnosis of septicemia in infants. The method involves measuring the levels of one or more biomarkers against predefined, respective threshold values and interpreting these levels to arrive at the diagnosis. Other techniques may introduce a preliminary step of identifying higher risk subjects, as well as the integration of such methods into the final diagnostic methodology. One aspect of a technique of this method may involve measuring one more cytokines to detect specific classes of infective organisms, such as Gram-negative bacteria. Another technique may involve a system that provides a sampling device to measure certain biomarkers and utilizes a computer processing device to interpret the levels of such markers in order to determine the specific class of infective organism or blood culture result. This system may provide a preliminary system to identify high risk individuals, and it may also incorporate such systems and their measures into the primary diagnostic system. The technique may also provides a computer program product for determining the presence of a specific class of infective organism and/or blood culture result in an infant, whereby computer logic implements the above methodology.

An aspect of an embodiment of the present invention provides a method, system and computer program product for, among other things, determining the presence of a specific class of infective organism and/or blood culture result in an infant. This method, system and computer program product may comprise: measuring the levels of certain biomarkers in a sample and evaluating these levels against a predefined metric to determine the presence of a specific class of infective organism or blood culture result in the infant. This method, system and computer program product can be used to detect the presence of classes of organisms such as, but not limited to, Gram-negative, Gram-positive, coagulase-negative staphylococci, and fungus; as well as identifying samples containing no such growth. Without wishing to be bound by any particular theory it is hypothesized that this method, system and computer program product can be used to detect the presence of classes of organisms such as, but not limited to, other bacteria and other pathogens, as well as viruses.

In an embodiment, the sample may be a blood sample. In another embodiment, the biomarkers measured may be cytokines. In yet another embodiment, the biomarkers may comprise at least one of the following cytokines: IL-6, IL-8, TNF-α, or G-CSF. Testing a sample for threshold levels of these biomarkers allows for improved detection of neonatal sepsis and identification of particular infective organisms and blood culture results.

In an aspect of embodiment of the present invention, the biomarker analysis described above may be prompted by a preliminary diagnostic step, such as measuring heart rate characteristics or other physiological measures. In another embodiment, the biomarker analysis, whether prompted by such a preliminary step or not, may also incorporate other diagnostic steps such as measuring heart rate characteristics or other physiological measures.

Still another aspect of an embodiment of the present invention involves a system and method for determining the presence of a specific class of infective organism and/or blood culture result in infants. This system and method may comprise: a sampling device for measuring the levels of one or more biomarkers in a sample and one or more computer processing devices configured for interpreting these biomarkers in order to detect a specific class of infective organism or blood culture result.

In an embodiment of this system and method, the sample is a blood sample. In another embodiment, the biomarkers measured are cytokines. In yet another embodiment, the biomarkers comprise at least one of the following cytokines: IL-6, IL-8, TNF-α, or G-CSF.

In another aspect of an embodiment of the present invention, the system described above also contains a preliminary diagnostic system, such as devices for measuring heart rate characteristics or other physiologic measures, which would identify subjects who were at higher than normal risk. In yet another embodiment, the diagnostic system, regardless of whether it includes a preliminary system for identifying high-risk subjects, also includes a device for measuring heart rate characteristics or other physiologic measures and incorporates such measures into its diagnostic analysis.

These and other objects, along with advantages and features of various aspects of embodiments of the invention disclosed herein, will be made more apparent from the description, drawings and claims that follow.

### BRIEF DESCRIPTION OF THE DRAWINGS

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIG. 1A is a box plot showing the distribution of G-CSF densities in samples in the SRO, CS, BCPS, and GNB groups.

FIG. 1B is a box plot showing the distribution of IL-1ra densities in samples in the SRO, CS, BCPS, and GNB groups.

FIG. 1C is a box plot showing the distribution of IL-8 densities in samples in the SRO, CS, BCPS, and GNB groups.

FIG. 1D is a box plot showing the distribution of TNF-α, densities in samples in the SRO, CS, BCPS, and GNB groups.

FIG. 1E is a box plot showing the distribution of IL-10 densities in samples in the SRO, CS, BCPS, and GNB groups.

FIG. 1F is a box plot showing the distribution of IL-6 densities in samples in the SRO, CS, BCPS, and GNB groups.

FIG. 1G is a box plot showing the distribution of IP-10 densities in samples in the SRO, CS, BCPS, and GNB groups.

FIG. 2A is a box plot showing the distribution of C-Reactive Protein densities in samples in the SRO, CS, BCPS, and GNB groups.

FIG. 2B is a box plot showing the distribution of cytokine scores in samples in the SRO, CS, BCPS, and GNB groups.

FIG. 3 is a hierarchical cluster analysis of cytokines levels in samples containing infective organisms.

FIG. 4A is a table showing GNB sensitivity, specificity, positive predictive value, and negative predictive value for several physiomarker and biomarker measures.

FIG. 4B is a table showing SRO sensitivity, specificity, positive predictive value, and negative predictive value for several physiomarker and biomarker measures.

FIG. 5 is a schematic block diagram for a system or related method of an embodiment of the present invention in whole or in part.

### DETAILED DESCRIPTION OF EXEMPLARY EMBODIMENTS

An aspect of an embodiment of the present invention provides, but is not limited thereto, a method (and related system and computer program product) for diagnosing a specific class of infective organism in infants. This method may involve first measuring the levels of one or more biochemical substances in a sample, then assessing these levels against a predetermined target value. This assessment is then interpreted to determine the presence of a specific class of infective organism or blood culture result. It should be appreciated that this method can involve measuring a single biomarker or several biomarkers, each with their own threshold values. Regarding an aspect of another embodiment of the present invention, the tested sample may be a blood sample. However, it should be noted that the sample can be any sample that is capable of being tested for the presence of the necessary biochemical substances. Furthermore, separate samples from the same infant might be tested during the course of a single diagnostic test. It should be appreciated that the biochemical substances may be circulating substances. Moreover, it should be appreciated that the biochemical substances may be non-circulating substances or intracellular substances.

Regarding another aspect of an embodiment of the invention, the assessment of the levels of one or more biochemical substances involves identifying and counting the number of substances whose levels are above or below a threshold value. In yet another embodiment of the invention, this counting yields a score that is then interpreted to detect a particular class of invective organism or blood culture result. It should be appreciated that such embodiments are merely examples, and other embodiments of the invention may utilizing various measuring metrics, scoring methods, and interpretive algorithms. For example, rather than assigning a score based on the number of biochemical substances that meet or fail to meet the threshold value, other embodiments might utilize a fluid scoring system that assesses the degree to which the level of one or more biochemical substances exceeds a target value.

Regarding an aspect of another embodiment of the present invention, the circulating substances measured in the samples may be cytokines. In yet another embodiment of the invention, the cytokines comprise at least one of the following: IL-6, IL-8, TNF-α, or G-CSF. Again, diagnostic methodology may examine the levels of a single cytokine or the levels of any number of cytokines in order to arrive at a diagnosis. One aspect of an embodiment of the invention involves counting the number of these cytokines that are above their respective threshold values in order to arrive at a “cytokine score,” which may lead directly to a diagnosis or be combined with other diagnostic measures to arrive at a final diagnosis. In this embodiment, a higher score indicates a higher probability of the particular diagnosis. However, it should be appreciated that this particular counting methodology is merely an illustrative example and is not meant to serve as a limitation.

Another aspect of an embodiment of the invention involves directing these diagnostic methods toward identifying at least one of the following classes of infective organism or blood culture result: Gram-negative, Gram-positive, coagulase-negative staphylococci, fungus, or no growth. For example, the presence of certain biomarkers above a predetermined threshold level might indicate that an infant is infected with Gram-negative bacteria, or the presence of a certain biomarker below a predetermined threshold level might indicate that an infant is in fact not septic. Again, these examples merely serve to illustrate how such a diagnostic method might be structured and is not intended to limit the invention. For instance, without wishing to be bound by any particular theory it is hypothesized that an embodiment may involves directing these diagnostic methods toward identifying at least one of the following classes of infective organism or blood culture result: other bacteria and other pathogens, as well as viruses.

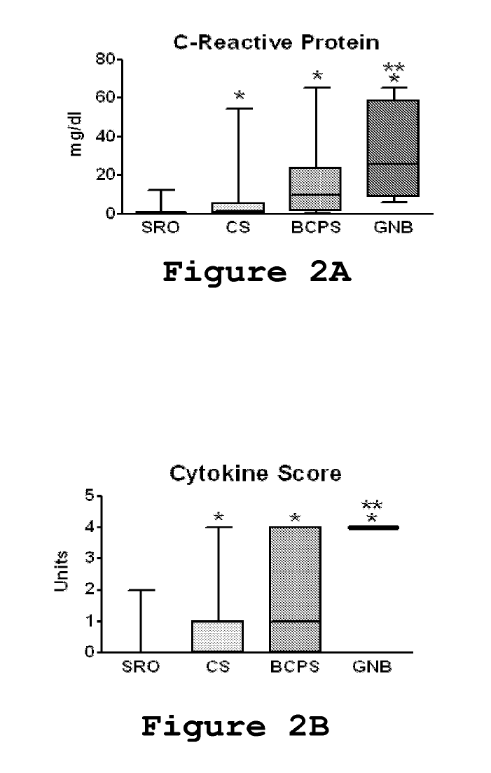

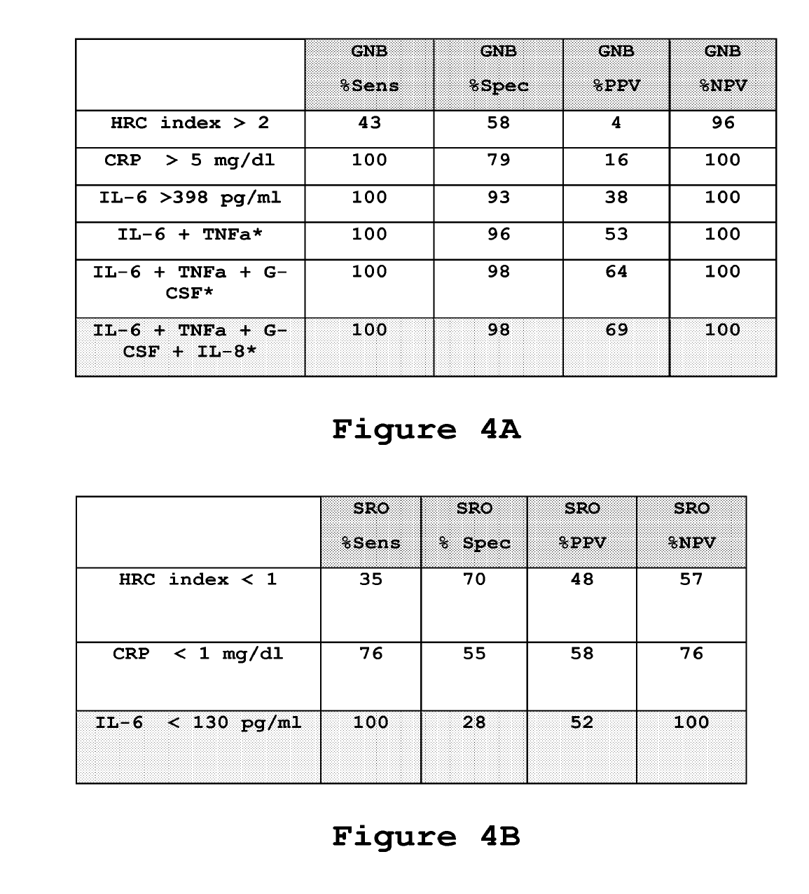

Turning to an aspect of an embodiment of the present invention, the measured biomarkers are IL-6, IL-8, TNF-α, and G-CSF; and the threshold values for these cytokines are about 400 pg/ml for IL-6, about 200 pg/ml for IL-8, about 1000 pg/ml for G-CSF, and about 32 pg/ml for TNF-α. FIG. 2B shows the results of a clinic study in which this methodology was evaluated for its sensitivity and predictive ability for several classifications of septicemia. It should be appreciated that the thresholds may be increased or decreased as desired or required. In this particular embodiment, for example, a sample that measures above these threshold values for all four cytokines would indicate Gram-negative bactermia (GNB) with 100% sensitivity and 69% positive predictive value, as shown in FIG. 4A. FIG. 4A also shows several other diagnostic methodologies that utilize one or more biomarkers to identify GNB patients. Again, these particular embodiments serve only as examples and are not intended to limit the scope of the invention.

Turning to an aspect of another embodiment of the present invention, the measured cytokine is IL-6, which is measured against a lower threshold of about 130 pg/ml. Under this methodology, samples measuring below this threshold indicate no growth with 100% sensitivity and 52% positive predictive value, as shown in FIG. 4B. Again, this embodiment is merely one example of how the present invention may be implemented. It should be appreciated that the thresholds may be increased or decreased as desired or required.

An aspect of embodiment of the present invention involves combining the above-described methodology with a preliminary step that identifies individuals who are at a higher than normal risk of having a particular infective organism or blood culture result. For example, one aspect of this embodiment involves utilizing heart rate characteristics (HRC) monitoring to identify infants that have a higher probability of having septicemia. HRC can be monitored on several types of devices. The signal may be obtained from a subject and recorded using devices or machinery known in the art, e.g., heart monitors, such as the heart rate characteristics index monitor (HeRO™, Medical Predictive Science Corporation, Charlottesville, Va.), Philips Intellivue, or GE Solar monitors. The recorded physiological signal may be further processed after it is recorded. Furthermore, it should be noted that HRC monitoring is merely one example of how such a preliminary step might be implemented. Still another embodiment of the invention combines this additional diagnostic step with the measuring of the biomarker levels in order to arrive at the particular diagnosis. It should be noted that even if this additional diagnostic measurement is incorporated into the biomarker interpretation, the method may or may not also utilize the preliminary step described above.

An aspect of an embodiment of the present invention involves a system for determining the presence of a specific class of infective organism and/or blood culture result in infants. This system includes a sampling device for measuring the levels of one or more biochemical substances in one or more samples, as well as one or more computer processing devices configured for assessing these levels against a target value and interpreting said assessment to determine the presence of a specific class of infective organism or blood culture result. In another embodiment of this system, the assessment involves counting the number of said one or more substances that are above or below a threshold value.

Regarding an aspect of an embodiment of the invention, at least one of the samples measured by the sampling device may be a blood sample. For one subject, the sampling device might examine a single blood sample, multiple blood samples, or a blood sample in addition to other types of samples.

Regarding an aspect of an embodiment of the invention, the circulating substances examined by the sampling device may include one or more cytokines. These cytokines can include IL-6, IL-8, TNF-α, and G-CSF. It should be appreciated that the sampling device could measure the levels of a single biomarker, or it could measure the levels of any combination of these biomarkers.

Regarding an aspect of an embodiment of the invention, the system may be directed at detecting at least one of the following infective organisms or blood culture results: Gram-negative, Gram-positive, coagulase-negative staphylococci, fungus, or no growth. A single system may be configured to provide one or more of these diagnoses at the same time.

Similar to the method described above, in an embodiment of the invention, the sampling device measures IL-6, IL-8, TNF-α, and G-CSF for threshold values of about 400 pg/ml for IL-6, about 200 pg/ml for IL-8, about 1000 pg/ml for G-CSF, and about 32 pg/ml for TNF-α. In this particular embodiment, for instance, a sample that measures above these threshold values for all four cytokines would indicate Gram-negative bactermia (GNB) with 100% sensitivity and 69% positive predictive value, as shown in FIG. 4A. The system might also be configured to examine other combinations of biomarkers to identify GNB or other classes of infective organisms. Again, these particular embodiments serve only as examples and are not intended to limit the scope of the invention. It should be appreciated that the thresholds may be increased or decreased as desired or required.

Regarding an aspect of an embodiment, the sampling device measures IL-6 for levels below a lower threshold of about 130 pg/ml. In this embodiment of the system, for instance, samples measuring below this threshold value indicate no growth with 100% sensitivity and 52% positive predictive value, as shown in FIG. 4B. Again, this embodiment is merely one example of how the system might be implemented. It should be appreciated that the thresholds may be increased or decreased as desired or required.

Other embodiments of the system may involve generating scores based on the number of biomarkers and/or physiomarkers that register above and/or below their respective threshold values. In such a system, a higher score (i.e. a greater number of biomarkers and physiomarkers that satisfy the threshold requirement) indicates a higher probability that the subject has a particular class of infective organism or blood culture result.

An aspect of an embodiment of the invention may involve incorporating a preliminary system for identifying subjects at higher than normal risk of having the specific class of infective organism or blood culture result. One example of such a system is an HRC monitoring system such as the devices mentioned above. The preliminary system could also involve a device configured to monitor or detect other physiologic measures. Beyond the presence of an HRC monitoring device and/or other devices for measuring physiologic symptoms, the system may also incorporate a computer processing device that is configured for interpreting these heart rate characteristics and/or other physiologic measures. Furthermore, other embodiments of the invention might incorporate such HRC monitors and/or physiologic measures into the primary computer processing device such that these measures are incorporated into the ultimate diagnostic metric rather than simply acting as preliminary “gatekeeper” systems.

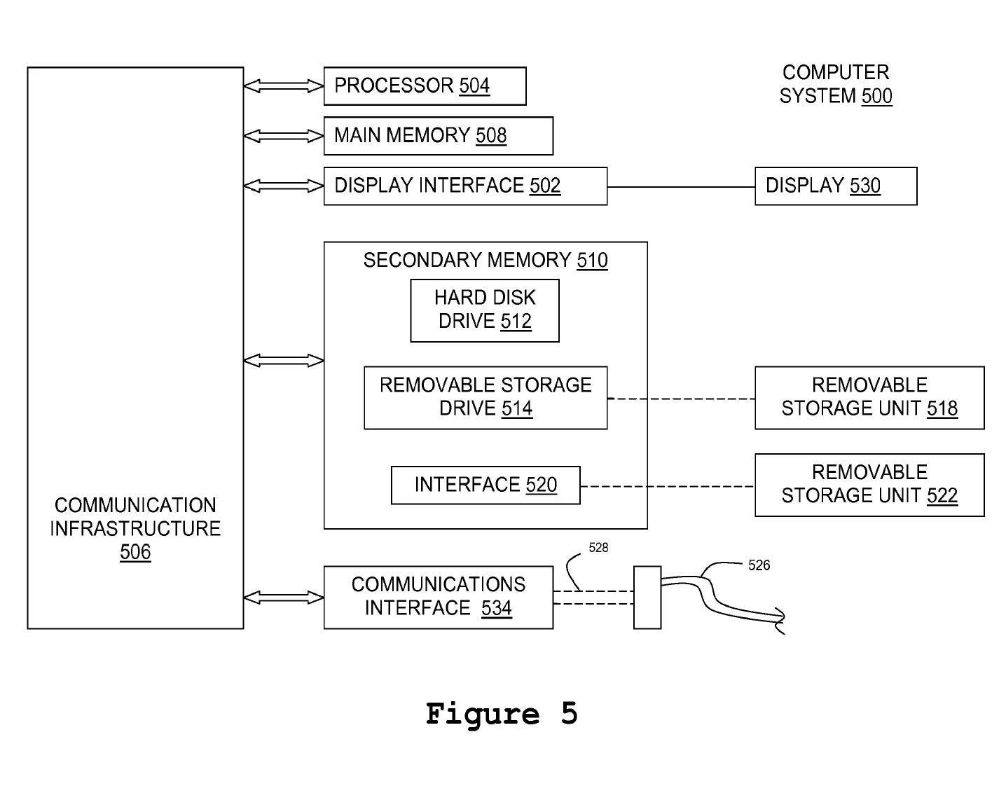

Turning to FIG. 5, FIG. 5 is a functional block diagram for a computer system 500 for implementation of an exemplary embodiment or portion of an embodiment of present invention. For example, a method or system of an embodiment of the present invention may be implemented using hardware, software or a combination thereof and may be implemented in one or more computer systems or other processing systems, such as personal digit assistants (PDAs) equipped with adequate memory and processing capabilities. In an example embodiment, the invention was implemented in software running on a general purpose computer 50 as illustrated in FIG. 5. The computer system 500 may includes one or more processors, such as processor 504. The Processor 504 is connected to a communication infrastructure 506 (e.g., a communications bus, cross-over bar, or network). The computer system 500 may include a display interface 502 that forwards graphics, text, and/or other data from the communication infrastructure 506 (or from a frame buffer not shown) for display on the display unit 530. Display unit 530 may be digital and/or analog.

The computer system 500 may also include a main memory 508, preferably random access memory (RAM), and may also include a secondary memory 510. The secondary memory 510 may include, for example, a hard disk drive 512 and/or a removable storage drive 514, representing a floppy disk drive, a magnetic tape drive, an optical disk drive, a flash memory, etc. The removable storage drive 514 reads from and/or writes to a removable storage unit 518 in a well known manner. Removable storage unit 518, represents a floppy disk, magnetic tape, optical disk, etc. which is read by and written to by removable storage drive 514. As will be appreciated, the removable storage unit 518 includes a computer usable storage medium having stored therein computer software and/or data.

In alternative embodiments, secondary memory 510 may include other means for allowing computer programs or other instructions to be loaded into computer system 500. Such means may include, for example, a removable storage unit 522 and an interface 520. Examples of such removable storage units/interfaces include a program cartridge and cartridge interface (such as that found in video game devices), a removable memory chip (such as a ROM, PROM, EPROM or EEPROM) and associated socket, and other removable storage units 522 and interfaces 520 which allow software and data to be transferred from the removable storage unit 522 to computer system 500.

The computer system 500 may also include a communications interface 524. Communications interface 124 allows software and data to be transferred between computer system 500 and external devices. Examples of communications interface 524 may include a modem, a network interface (such as an Ethernet card), a communications port (e.g., serial or parallel, etc.), a PCMCIA slot and card, a modem, etc. Software and data transferred via communications interface 524 are in the form of signals 528 which may be electronic, electromagnetic, optical or other signals capable of being received by communications interface 524. Signals 528 are provided to communications interface 524 via a communications path (i.e., channel) 526. Channel 526 (or any other communication means or channel disclosed herein) carries signals 528 and may be implemented using wire or cable, fiber optics, blue tooth, a phone line, a cellular phone link, an RF link, an infrared link, wireless link or connection and other communications channels.

In this document, the terms “computer program medium” and “computer usable medium” are used to generally refer to media or medium such as various software, firmware, disks, drives, removable storage drive 514, a hard disk installed in hard disk drive 512, and signals 528. These computer program products (“computer program medium” and “computer usable medium”) are means for providing software to computer system 500. The computer program product may comprise a computer useable medium having computer program logic thereon. The invention includes such computer program products. The “computer program product” and “computer useable medium” may be any computer readable medium having computer logic thereon.

Computer programs (also called computer control logic or computer program logic) are may be stored in main memory 508 and/or secondary memory 510. Computer programs may also be received via communications interface 524. Such computer programs, when executed, enable computer system 500 to perform the features of the present invention as discussed herein. In particular, the computer programs, when executed, enable processor 504 to perform the functions of the present invention. Accordingly, such computer programs represent controllers of computer system 500.

In an embodiment where the invention is implemented using software, the software may be stored in a computer program product and loaded into computer system 500 using removable storage drive 514, hard drive 512 or communications interface 524. The control logic (software or computer program logic), when executed by the processor 504, causes the processor 504 to perform the functions of the invention as described herein.

In another embodiment, the invention is implemented primarily in hardware using, for example, hardware components such as application specific integrated circuits (ASICs). Implementation of the hardware state machine to perform the functions described herein will be apparent to persons skilled in the relevant art(s).

In yet another embodiment, the invention is implemented using a combination of both hardware and software.

In an example software embodiment of the invention, the methods described above may be implemented in SPSS control language or C++ programming language, but could be implemented in other various programs, computer simulation and computer-aided design, computer simulation environment, MATLAB, or any other software platform or program, windows interface or operating system (or other operating system) or other programs known or available to those skilled in the art.

It should also be appreciated that the exact manner of measuring the levels of one or more biochemical substances and the subsequent analysis can be accomplished by any number of techniques. For example, it may be achieved by the common paradigm whereby samples are taken in person and the samples are analyzed locally or are physically transferred to other facilities where they can be tested and analyzed. However, it may also be achieved by incorporating a “telemedicine” paradigm whereby, at one or more points during the process, information is transferred over a wired or wireless data communications network to a remote location where subsequent analysis or other processing may take place. For example, an aspect of embodiment of the invention may involve electronically transferring the results of sample measurement (such as cytokine levels) over a data communications network to a remote location where subsequent assessment and/or analysis can take place. Such utilization of telecommunications networks may occur during any step in the process and may be utilized at a single or multiple points. Likewise, telecommunications networks may be incorporated into any part of the system.

Furthermore, information can be displayed at any point during the process, or at any point in the system, in any number of ways. For example, readings and data may be received and/or displayed by the user, clinician, physician, technician, patient or the like by hard copy (e.g., paper), visual graphics, audible signals (such as voice or tones, for example), or any combination thereof. Additionally, any measurements, assessment, analysis, secondary information, diagnosis, reading, data, or discussion may be reduced to hard copy (e.g., paper) or computer storage medium at any point during the process (or system).

### EXAMPLES

Practice of an aspect of an embodiment (or embodiments) of the invention will be still more fully understood from the following examples and experimental results, which are presented herein for illustration only and should not be construed as limiting the invention in any way.

### Experimental Results and Examples Set No. 1

Remnant plasma was collected from NICU patients greater than 3 days old undergoing blood culture for suspected sepsis. Patients of all gestational ages were included. Samples were collected over an 18 month period at 2 centers (University of Virginia, “Center A”, and Wake Forest University, “Center B”). Birth weight, gestational age, duration of antibiotic therapy, and blood culture results were recorded. Samples were classified as sepsis ruled out (negative blood culture and antibiotics for <5 days), clinical sepsis (negative blood culture but antibiotics continued ≥5 days), blood culture-positive sepsis (a positive blood culture in a patient with signs and symptoms of sepsis), or Gram-negative bacteremia (a positive blood culture for Gram-negative bacteria in a patient with signs and symptoms of sepsis). All patient information was deidentified and the Institutional Review Boards of each institution approved collection of remnant plasma samples with waiver of consent.

Plasma samples were obtained from EDTA-containing tubes which had been obtained for complete blood count at or near (within 6 hours of) the time of blood culture. Following storage at 4° C. for less than 24 hours, blood was centrifuged and plasma stored at −80° C. until batch analysis for cytokines.

Seven cytokines were measured using a multiplex antibody-coated bead array with dual laser fluorometric detection (Milliplex, Millipore, Billerica, Mass.). Analytes included interleukin-6 (IL-6), IL-8, IL-10, IL-1 receptor antagonist, interferon gamma-inducible protein-10 (IP-10), tumor necrosis factor-alpha (TNF-α), and granulocyte colony-stimulating factor (G-CSF). Samples were diluted 1:4 and assayed in duplicate according to the manufacturer's instructions. Limit of detection was 3.2 pg/ml.

C-reactive protein (CRP) was measured by immunoassay at the time of blood culture at Center B and at the end of the study, if sufficient plasma remained after cytokine testing, at Center A.

The FDA-cleared heart rate characteristics index monitor (HeRO™, Medical Predictive Science Corporation, Charlottesville, Va.) takes electrocardiogram data from existing ICU monitors and calculates the standard deviation of normal RR intervals (SDNN), sample entropy, and sample asymmetry for each epoch of 4096 heart beats. These three characteristics are used to generate an HRC index which is the fold increase in risk that a patient will be diagnosed with clinical or culture-proven sepsis in the next 24 hours. The HeRO monitor continuously displays the HRC index which is calculated every hour and reflects heart rate variability and decelerations over the previous 12 hours. For the purpose of this study, maximum HRC index in the 12 hours preceding blood culture was recorded.

Plasma samples for this study were collected during a randomized clinical trial in which very low birth weight infants underwent continuous monitoring of the HRC index and were randomized to having their HRC index displayed to clinicians or not displayed. HRC index data for this study were collected after completion of the randomized clinical trial. Patients >1500 grams birth weight had HRC index monitored and visible to clinicians at Center A but not at Center B. Clinicians were educated about HRC monitoring but no course of action was prescribed for abnormal or changing HRC index.

Cytokines, CRP, and HRC index in the four groups SRO, CS, BCPS and GNB were compared by Kruskal-Wallis analysis followed by Dunn's multiple comparison tests. In comparing GNB to BCPS, analysis was performed both with and without the GNB samples included in the BCPS group. Correlation of HRC index and individual cytokines was assessed using Spearman correlation coefficients (GraphPad Prism version 4, San Diego, Calif.). A p value <0.05 was considered statistically significant.

Hierarchical cluster analysis was performed on the seven cytokines in samples associated with a positive blood culture (MATLAB Bioinformatics Toolbox, MathWorks, Natick, Mass.). For each cytokine, thresholds were established to give 100% sensitivity and negative predictive value for Gram-negative bacteremia. A separate analysis was performed to determine thresholds with 100% sensitivity and negative predictive value for sepsis ruled-out. Using these thresholds, all 127 possible combinations of the 7 cytokines were tested to determine the combination with maximum positive predictive value for either GNB or SRO.

226 plasma samples were obtained near the time of blood culture from 163 patients. Gestational age was 28.7±4.7 weeks and birth weight was 1311±861 grams (mean±SD). Samples were classified as sepsis ruled out (SRO, negative blood culture and antibiotics for <5 days, n=98), clinical sepsis (CS, negative blood culture but antibiotics continued ≥5 days, n=95), blood culture positive sepsis (BCPS, n=33), or Gram-negative bacteremia (GNB, n=9). Organisms in the positive blood cultures were coagulase-negative staphylococcus species (CoNS, n=16), Staphylococcus aureus (4), Enterococcus fecalis (3), Escherichia coli (3), Klebsiella species (3), Pseudomonas aeruginosa (1), Enterobacter cloacae (1), Raoultella ornithinolytica (1), and Candida species (2). One sample yielded two organisms (CoNS and Candida).

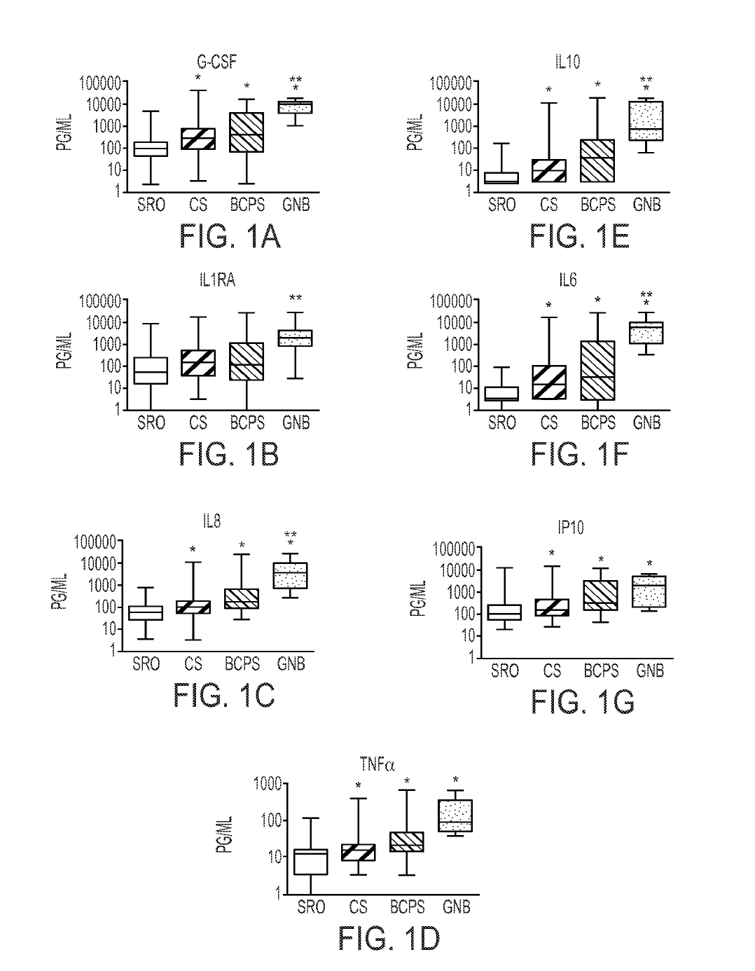

FIGS. 1A-1G show box plots describing the distribution of cytokine levels in each of the four sample groups. Seven cytokines were analyzed in 226 plasma samples from NICU patients >3 days old with suspected sepsis, subsequently classified as sepsis ruled out (SRO, n=98), clinical sepsis (CS n=95), blood culture-positive sepsis (BCPS n=33), or Gram-negative bacteremia (GNB, n=9). In these figures, the horizontal line within the box is the median, the boundaries of the box are 25^{th} and 75^{th} percentile, and the whiskers are minimum and maximum values. Six cytokines (all except IL-1ra) were significantly higher in patients with clinical or blood culture-positive sepsis compared with sepsis ruled out (*p<0.05 versus SRO), and samples associated with Gram-negative bacteremia had significantly higher levels of six cytokines (all except IP-10) compared with those associated with Gram-positive bacteria or Candida (all p<0.05). There were no significant differences in any cytokine in patients with clinical sepsis versus blood culture-positive sepsis.

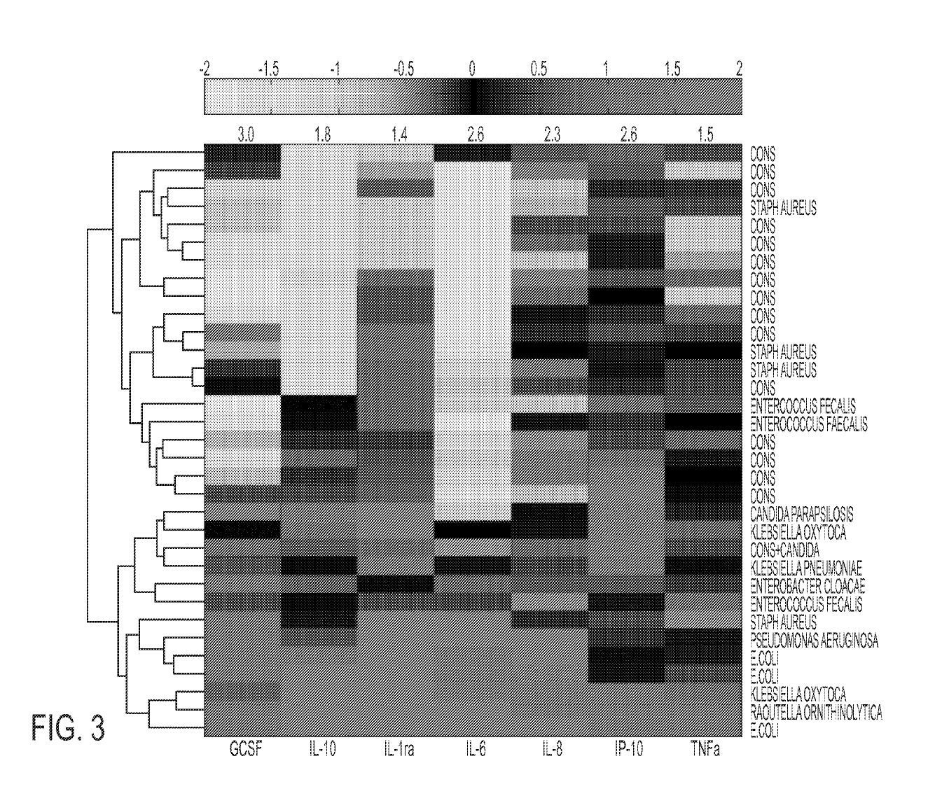

FIG. 3 shows the hierarchical cluster analysis of cytokines from the 33 plasma samples associated with a positive blood culture, showed clustering of Gram-negative organisms among the samples with the highest cytokine levels. Thresholds for each cytokine were established to identify all cases of GNB, then all possible combinations of the seven cytokines were tested to determine the optimal combination for identifying all GNB cases. The 127 combinations had 100% sensitivity (by design), with positive predictive values ranging from 5 to 69% (median=53%). There were 8 combinations that achieved the maximum performance of 69% PPV, and only one combination included only 4 cytokines. Based on this analysis, the following four cytokines and thresholds were used to generate a cytokine score: G-CSF (1000 pg/ml), IL-6 (400 pg/ml), IL-8 (200 pg/ml), and TNF-α (32 pg/ml). Assigning a 1 or 0 based on these thresholds, a cytokine score of 4 had 100% sensitivity and negative predictive value for identifying patients with Gram-negative bacteremia, with 69% positive predictive value, as shown in FIG. 4A. While approaches that result in empirical sensitivities of 100% necessarily overestimate performance, this is a reasonable way to identify optimal thresholds and combinations of cytokines in data with a large separation among groups.

Four samples with a cytokine score of 4 were not associated with Gram-negative bacteremia, and in each case the patient was very ill. One had E. coli pneumonia and the other three had severe gastrointestinal pathology (two cases of necrotizing enterocolitis and one case of gastric perforation with peritonitis).

Using the same strategy as that described for GNB, we tested cytokine thresholds (individual and combination) for identifying the 98 cases of “sepsis ruled out”. As shown in FIG. 4B, the best performing individual cytokine was IL-6<130 pg/ml which gave 100% sensitivity and 52% NPV for SRO. Adding any other cytokine to IL-6, alone or in combination, did not result in a higher NPV.

CRP was measured on 177 of the 226 samples (78%). There were similar proportions of samples with CRP available for analysis in the four groups SRO, CS, BCPS, and GNB (75-82%). CRP was significantly correlated with each of the seven cytokines studied (IL-6 r=0.52, G-CSF r=0.50, IL-10 r=0.46, IL-8 r=0.39, IP-10 r=0.39, TNF-α r=0.29, IL-1ra r=0.21, all p<0.01). There was no significant correlation of CRP with the HRC index. As shown in FIG. 2A, CRP was significantly higher in clinical and blood culture-positive sepsis and Gram-negative bacteremia than in sepsis ruled out, and in GNB versus BCPS. This was true whether the 9 GNB samples were compared with all 33 BCPS or with only the 24 non-GNB cases of septicemia.

The HRC index was continuously monitored on all patients at Center A and on very low birthweight infants at Center B. Of the 226 samples for cytokine analysis, 188 had an associated HRC index available for analysis. For the other samples, either the patient was at Center B and not VLBW or the HRC index was not available near the time of sample acquisition.

The HRC index was significantly correlated with plasma levels of IL-8 and IL-1ra (IL-8 r=0.20, p=0.004; IL-1ra r=0.30, p<0.0001), but not with the other five cytokines studied (p value range 0.06 for IL-6 to 0.97 for TNF-α) or with the Cytokine Score (p=0.1775). The HRC index was not significantly different in patients with sepsis ruled out, clinical sepsis, blood culture positive sepsis, or Gram-negative bacteremia (all p>0.05). As shown in FIGS. 4A and 4B, HRC index >2 had 43% sensitivity for GNB and HRC index<1 had 35% sensitivity for SRO. Since 79 samples were obtained from infants whose HRC index was displayed to clinicians, which may have impacted decisions about obtaining blood cultures and duration of antibiotic therapy, the 147 samples from patients whose HRC index was not displayed to clinicians were analyzed separately, and again no significant differences among the groups were found (data not shown).

Thus, in this study of patients with clinically suspected sepsis, heart rate characteristics measurements did not further discriminate between those with sepsis ruled out and those with clinical or blood culture positive sepsis, whereas cytokines performed well. Six of the seven cytokines analyzed were significantly higher in patients with clinical or blood culture positive sepsis compared with those with sepsis ruled out and were higher in patients with Gram-negative bacteremia compared with other septicemia. A 4-cytokine combination was identified which identified all patients with Gram-negative bacteremia with reasonable positive predictive value.

By including four analytes to assign a cytokine score (G-CSF, IL-6, IL-8, and TNF-α), all 9 cases of Gram-negative bacteremia were identified with a false positive rate of only 31%. Higher cytokine levels have been reported in plasma of adults with Gram-negative compared with Gram-positive bacteremia. Endotoxin on Gram-negative organisms has been shown to induce greater cytokine production by leukocytes compared with toxins on Gram-positive bacteria, and this likely accounts, at least in part, for the higher incidence of septic shock, multi-organ dysfunction, and death in patients with Gram-negative septicemia.

IL-6 has been identified as a promising biomarker in other studies of neonates with suspected sepsis, and this study also showed that, of the seven cytokines analyzed, IL-6 had the best diagnostic accuracy. In fact, no cytokine combination had better performance than IL-6 alone at identifying patients undergoing blood culture in whom sepsis was subsequently ruled out. With only 52% positive predictive accuracy (i.e. 48% of samples with IL-6<130 pg/ml occurring in patients with a subsequent diagnosis of either clinical of blood culture-positive sepsis), this test would likely not be useful to clinicians in making a decision not to initiate antibiotic therapy in a patient with significant sepsis-like symptoms. However, in a patient with equivocal signs or symptoms, a low plasma level of IL-6 might serve as a useful adjunct test to reinforce a clinician's decision not to initiate antibiotic therapy.

While cytokines were only assayed at the time of blood culture, other studies have shown that additional measurements of biomarkers a day later can increase the diagnostic accuracy of these assays. This is especially true of acute phase proteins such as C-reactive protein which rises 6-12 hours after cytokines are released in the circulation in response to bacteremia. A C-reactive protein threshold set to detect all cases of Gram-negative bacteremia at the time of blood culture was also found to have a very low positive predictive value compared to individual cytokines. While follow-up assays such as CRP may be useful for decisions about early discontinuation of antibiotics, highly sensitive assays available “on demand” at the time of blood culture are essential for initial therapeutic decisions.

The mean HRC index in the group of patients with sepsis ruled out was comparable to those with clinical or blood culture positive sepsis. It should be noted that HRC index monitoring was developed to detect subclinical phases of illnesses like sepsis, by which time HRC monitoring had already served its purpose. This is reflected in the relatively high mean HRC index of >2 in the study sample, compared with a mean overall HRC index of preterm NICU patients of <1.

A rise in the HRC index can indicate sepsis but it also may occur in non-septic conditions such as acute respiratory decompensation or severe apnea. Addition of a biomarker screen at the time of a rise in the HRC index over the patient's baseline could assist in decisions about evaluation for sepsis or initiation of empiric antibiotic therapy.

### Additional Example Sets

Example 1 includes a method of determining the presence of a specific class of infective organism and/or blood culture result in an infant, wherein said method comprises: measuring the levels of one or more biochemical substances in one or more samples; assessing levels of said one or more biochemical substances against a target value; and interpreting said assessment to provide said determination of the presence of a specific class of infective organism or blood culture result in the infant.

Example 2 may optionally include the method of example 1, wherein said assessment comprises: counting the number of said one or more biochemical substances whose levels are above or below a threshold value.

Example 3 may optionally include the method of example 1 (as well as subject matter of one or more of any combination of examples 1-2), wherein: at least one of said one or more samples is a blood sample.

Example 4 may optionally include the method of example 1 (as well as subject matter of one or more of any combination of examples 1-3), wherein: said one or more biochemical substances comprises one or more circulating substances.

Example 5 may optionally include the method of example 4 (as well as subject matter of one or more of any combination of examples 1-4), wherein: one or more of said one or more circulating substances are cytokines.

Example 6 may optionally include the method of example 1 (as well as subject matter of one or more of any combination of examples 1-5), wherein: said one or more biochemical substances comprises one or more non-circulating substances or one or more intracellular substances.

Example 7 may optionally include the method of example 5 (as well as subject matter of one or more of any combination of examples 1-6), wherein said cytokines comprise at least one of the following: IL-6; IL-8; TNF-α; or G-CSF.

Example 8 may optionally include the method of example 1 (as well as subject matter of one or more of any combination of examples 1-7), wherein said class of infective organism or blood culture result comprises at least one of the following: Gram-negative; Gram-positive; coagulase-negative staphylococci; fungus; viruses; bacteria; pathogens; or no growth.

Example 9 may optionally include the method of example 2 (as well as subject matter of one or more of any combination of examples 1-8), wherein: said class of infective organism is Gram-negative; and said threshold value is about 400 pg/ml for IL-6, about 200 pg/ml for IL-8, about 1000 pg/ml for G-CSF, and about 32 pg/ml for TNF-α.

Example 10 may optionally include the method of example 7 (as well as subject matter of one or more of any combination of examples 1-9), wherein: said class of infective organism is Gram-negative; and said target value is about 400 pg/ml for TL-6, about 200 pg/ml for IL-8, about 1000 pg/ml for G-CSF, and about 32 pg/ml for TNF-α.

Example 11 may optionally include the method of example 2 (as well as subject matter of one or more of any combination of examples 1-10), wherein: said blood culture result is no growth; and said threshold value is less than about 130 pg/ml for IL-6.

Example 12 may optionally include the method of example 7 (as well as subject matter of one or more of any combination of examples 1-11), wherein: said blood culture result is no growth; and said target value is about 130 pg/ml for IL-6.

Example 13 may optionally include the method of example 1 (as well as subject matter of one or more of any combination of examples 1-12), wherein said interpreting comprises: assigning a score based on said levels such that a higher score indicates a higher probability of the presence of said specific class of infective organism or blood culture result.

Example 14 may optionally include the method of example 1 (as well as subject matter of one or more of any combination of examples 1-13), further comprising a preliminary step of identifying subjects at higher than normal risk of having said specific class of infective organism or blood culture result.

Example 15 may optionally include the method of example 14 (as well as subject matter of one or more of any combination of examples 1-14), wherein said preliminary step comprises: measuring heart rate characteristics or other physiologic measures.

Example 16 may optionally include the method of example 1 (as well as subject matter of one or more of any combination of examples 1-15), further comprising: measuring heart rate characteristics or other physiologic measures; and wherein said interpreting incorporates analysis of said heart rate characteristics or other physiologic measures.

Example 17 includes a system for determining the presence of a specific class of infective organism and/or blood culture result in infants, wherein said system comprises: a sampling device for measuring the levels of one or more biochemical substances in one or more samples; one or more computer processing devices configured for assessing levels of said one or more biochemical substances against a target value; and interpreting said assessment to provide said determination of the presence of a specific class of infective organism or blood culture result in the infant.

Example 18 may optionally include the system of example 17 (as well as subject matter of one or more of any combination of examples 1-16), wherein said assessment comprises: counting the number of said one or more biochemical substances whose levels are above or below a threshold value.

Example 19 may optionally include the system of example 17 (as well as subject matter of one or more of any combination of examples 1-18), wherein: at least one of said one or more samples is a blood sample.

Example 20 may optionally include the system of example 17 (as well as subject matter of one or more of any combination of examples 1-19), wherein: said one or more biochemical substances comprises one or more circulating substances.

Example 21 may optionally include the system of example 20 (as well as subject matter of one or more of any combination of examples 1-20), wherein: one or more of said one or more circulating substances are cytokines.

Example 22 may optionally include the system of example 17 (as well as subject matter of one or more of any combination of examples 1-21), wherein: said one or more biochemical substances comprises one or more non-circulating substances or one or more intracellular substances.

Example 23 may optionally include the system of example 21 (as well as subject matter of one or more of any combination of examples 1-22), wherein said cytokines comprise at least one of the following: IL-6; IL-8; TNF-α; or G-CSF.

Example 24 may optionally include the system of example 17 (as well as subject matter of one or more of any combination of examples 1-23), wherein said class of infective organism or blood culture result comprises at least one of the following: gram-negative; gram-positive; coagulase-negative staphylococci; fungus; viruses; bacteria; pathogens; or no growth.

Example 25 may optionally include the system of example 18 (as well as subject matter of one or more of any combination of examples 1-24), wherein: said class of infective organism is Gram-negative; and said threshold value is about 400 pg/ml for IL-6, about 200 pg/ml for IL-8, about 1000 pg/ml for G-CSF, and about 32 pg/ml for TNF-α.

Example 26 may optionally include the system of example 23 (as well as subject matter of one or more of any combination of examples 1-25), wherein: said class of infective organism is Gram-negative; and said target value is about 400 pg/ml for IL-6, about 200 pg/ml for IL-8, about 1000 pg/ml for G-CSF, and about 32 pg/ml for TNF-α.

Example 27 may optionally include the system of example 18 (as well as subject matter of one or more of any combination of examples 1-26), wherein: said blood culture result is no growth; and said threshold value is less than about 130 pg/ml for IL-6.

Example 28 may optionally include the system of example 23 (as well as subject matter of one or more of any combination of examples 1-27), wherein: said blood culture result is no growth; and said target value is about 130 pg/ml for IL-6.

Example 29 may optionally include the system of example 17 (as well as subject matter of one or more of any combination of examples 1-28), wherein said interpreting comprises: assigning a score based on said levels such that a higher score indicates a higher probability of the presence of said specific class of infective organism or blood culture result.

Example 30 may optionally include the system of example 17 (as well as subject matter of one or more of any combination of examples 1-29), further comprising: a preliminary system for identifying subjects at higher than normal risk of having said specific class of infective organism or blood culture result.

Example 31 may optionally include the e system of example 30 (as well as subject matter of one or more of any combination of examples 1-30), wherein said preliminary system comprises: a measuring device for measuring heart rate characteristics or other physiologic measures; and a computer processing device configured for interpreting said heart rate characteristics or other physiologic measures.

Example 32 may optionally include the system of example 17 (as well as subject matter of one or more of any combination of examples 1-31), further comprising: a measuring device for measuring heart rate characteristics or other physiologic measures; and wherein said interpreting incorporates analysis of said heart rate characteristics or other physiologic measures.

Example 33 includes a computer program product comprising a computer useable medium having a computer program logic for enabling at least one processor in a computer system determining the presence of a specific class of infective organism and/or blood culture result in an infant, said computer logic comprising: measuring the levels of one or more biochemical substances in a sample; identifying and counting the number of said biochemical substances whose levels are above a threshold value; and interpreting said measures of said one or more circulating substances to provide said determination of the presence of a specific class of infective organism or blood culture result in the infant.

Example 34 may optionally include the computer program product of example 33 (as well as subject matter of one or more of any combination of examples 1-32), wherein said assessment comprises: counting the number of said one or more biochemical substances whose levels are above or below a threshold value.

Example 35 may optionally include the computer program product of example 33 (as well as subject matter of one or more of any combination of examples 1-34), wherein: said sample is a blood sample.

Example 36 may optionally include the computer program product of example 33 (as well as subject matter of one or more of any combination of examples 1-35), wherein: said one or more biochemical substances comprises one or more circulating substances.

Example 37 may optionally include the computer program product of example 36 (as well as subject matter of one or more of any combination of examples 1-36), wherein: one or more of said one or more circulating substances are cytokines.

Example 38 may optionally include the computer program product of example 33 (as well as subject matter of one or more of any combination of examples 1-37), wherein: said one or more biochemical substances comprises one or more non-circulating substances or one or more intracellular substances.

Example 39 may optionally include the computer program product of example 37 (as well as subject matter of one or more of any combination of examples 1-38), wherein said cytokines comprise at least one of the following: IL-6; IL-8; TNF-α; or G-CSF.

Example 40 may optionally include the computer program product of example 33 (as well as subject matter of one or more of any combination of examples 1-39), wherein said class of infective organism or blood culture result comprises at least one of the following: gram-negative; gram-positive; coagulase-negative; staphylococci; fungus; viruses; bacteria; pathogens; or no growth.

Example 41 may optionally include the computer program product of example 33 (as well as subject matter of one or more of any combination of examples 1-40), further comprising a preliminary step of identifying subjects at higher than normal risk of having said specific class of infective organism or blood culture result.

Example 42 may optionally include the computer program product of example 41 (as well as subject matter of one or more of any combination of examples 1-41), wherein said preliminary step comprises: measuring heart rate characteristics or other physiologic measures.

Example 43 may optionally include the computer program product of example 33 (as well as subject matter of one or more of any combination of examples 1-42), further comprising: measuring heart rate characteristics or other physiologic measures; and wherein said interpreting incorporates analysis of said heart rate characteristics or other physiologic measures.

The devices, systems, compositions, structures, computer program products, and methods of various embodiments of the invention disclosed herein may utilize aspects disclosed in the following references, applications, publications and patents and which are hereby incorporated by reference herein in their entirety:
1. U.S. patent application Ser. No. 12/866,056, filed Aug. 4, 2010, entitled “System, Method and Computer Program Product for Detection of Changes in Health Status and Risk of Imminent Illness”
2. U.S. patent application Ser. No. 12/724,162, filed Mar. 15, 2010, entitled “Method, System and Computer Program Method for Detection of Pathological Fluctuations of Physiological Signals to Diagnose Human Illness”, (publication mo. US 2010/0234748, Sep. 16, 2010.
3. U.S. patent application Ser. No. 12/594,842, filed Oct. 6, 2009, entitled “Method, System and Computer Program Product for Non-Invasive Classification of Cardiac Rhythm”, (publication no. US2010/0056940, Mar. 4, 2010.
4. International Patent Application Serial No. PCT/US2009/033082, filed Feb. 4, 2009, entitled “System, Method and Computer Program Product for Detection of Changes in Health Status and Risk of Imminent Illness”, (publication no. WO2009/100133).
5. International Patent Application Serial No. PCT/US2008/060021, filed Apr. 11, 2008, entitled “Method, System and Computer Program Product for Non-Invasive Classification of Cardiac Rhythm”, (publication no. WO2008/128034, Oct. 23, 2008).
6. U.S. patent application Ser. No. 10/545,257, filed Aug. 10, 2005, entitled “Quantitative Fetal Heart Rate and Cardiotocographic Monitoring System and Related Method Thereof”, (publication no. US2006/0074329, Apr. 6, 2006), U.S. Pat. No. 7,519,417, Ferguson, et al., issued Apr. 14, 2009.
7. U.S. patent application Ser. No. 11/000,630, filed Dec. 1, 2004, entitled “Method and “Apparatus for the Early Diagnosis of Subacute, Potentially Catastrophic Illness”, (publication no. US2005/137484, Jun. 23, 2005), U.S. Pat. No. 7,774,050, Griffin, et al., issued Aug. 10, 2010.
8. International Patent Application Serial No. PCT/US2004/004113, filed Feb. 12, 2004, entitled “Quantitative Fetal Heart Rate and Cardiotocographic Monitoring System and Related Method Thereof”, (publication no. WO2004/072822, Aug. 26, 2004).
9. International Patent Application Serial No. PCT/US2002/005676, filed Feb. 27, 2002, entitled “Method and Apparatus for the Early Diagnosis of Subacute, Potentially Catastrophic Illness”, (publication no. WO2002/067776, Sep. 6, 2002.
10. U.S. patent application Ser. No. 10/069,674, filed Feb. 22, 2002, entitled “Method and “Apparatus for Predicting the Risk of Hypoglycemia”, U.S. Pat. No. 6,923,763, Kovatchev, et al., issued Aug. 2, 2005.
11. U.S. patent application Ser. No. 09/793,653, filed Feb. 27, 2001, entitled “Method and “Apparatus for the Early Diagnosis of Subacute, Potentially Catastrophic Illness”, (publication no. US 2002/0052557 A1).
12. U.S. patent application Ser. No. 09/770,653, filed Jan. 29, 2001, entitled “Method for the Early Diagnosis of Subacute, Potentially Catastrophic Illness”, (publication no. US2003/0100841, May 29, 2003), U.S. Pat. No. 6,856,831, Griffin, et al., issued Feb. 15, 2005.
13. U.S. patent application Ser. No. 09/668,309, filed Sep. 25, 2000, entitled “Method and Apparatus for the Early Diagnosis of Subacute, Potentially Catastrophic Illness”, U.S. Pat. No. 6,330,469, Griffin, et al., issued Dec. 11, 2001.
14. International Patent Application Serial No. PCT/US00/22886, filed Aug. 21, 2000, entitled “Method and Apparatus for Predicting the Risk of Hypoglycemia”, (publication no. WO2001/13786, Mar. 1, 2001).
15. International Patent Application Serial No. PCT/US99/05687, filed Mar. 17, 1999, entitled “Method and Apparatus for the Early Diagnosis of Subacute, Potentially Catastrophic Illness”, (publication no. WO99/47040, Sep. 23, 1999).
16. U.S. patent application Ser. No. 09/271,279, filed Mar. 17, 1999, entitled “Method and Apparatus for the Early Diagnosis of Subacute, Potentially Catastrophic Illness”, U.S. Pat. No. 6,216,032, Griffin, et al., issued Apr. 10, 2001.
17. U.S. Patent Application Publication No. US 2009/0297474 A1, Kelleher, et al., “Method for Detecting or Monitoring Sepsis by Analysing Cytokine MRNA Expression Levels”, Dec. 3, 2009.
18. U.S. Pat. No. 7,767,395 B2, Garrett et al., “Diagnosis of Sepsis”, Aug. 3, 2010.
19. U.S. Pat. No. 7,645,573 B2, Ivey et al., “Diagnosis of Sepsis or SIRS Using Biomarker Profiles, Jun. 12, 2010.
20. U.S. Pat. No. 6,251,598 B1, di Giovine et al., “Methods for Diagnosing Sepsis”, Jun. 26, 2001.
21. Ng, P. C., S. H. Cheng, et al. (1997). “Diagnosis of late onset neonatal sepsis with cytokines, adhesion molecule, and C-reactive protein in preterm very low birthweight infants.” Arch Dis Child Fetal Neonatal Ed 77(3): F221-227.
22. Ng, P. C., K. Li, et al. (2006). “Early prediction of sepsis-induced disseminated intravascular coagulation with interleukin-10, interleukin-6, and RANTES in preterm infants.” Clin Chem 52(6): 1181-1189.
23. Ng, P. C., K. Li, et al. (2007). “IP-10 is an early diagnostic marker for identification of late-onset bacterial infection in preterm infants.” Pediatr Res 61(1): 93-98.
24. Gonzalez, B. E., C. K. Mercado, et al. (2003). “Early markers of late-onset sepsis in premature neonates: clinical, hematological and cytokine profile.” J Perinat Med 31(1): 60-68.
25. Laborada, G., M. Rego, et al. (2003). “Diagnostic value of cytokines and C-reactive protein in the first 24 hours of neonatal sepsis.” Am J Perinatol 20(8): 491-501.
26. Sherwin, C., R. Broadbent, et al. (2008). “Utility of interleukin-12 and interleukin-10 in comparison with other cytokines and acute-phase reactants in the diagnosis of neonatal sepsis.” Am J Perinatol 25(10): 629-636.
27. Kuster, H., M. Weiss, et al. (1998). “Interleukin-1 receptor antagonist and interleukin-6 for early diagnosis of neonatal sepsis 2 days before clinical manifestation.” Lancet 352(9136): 1271-1277.
28. Kingsmore, S. F., N. Kennedy, et al. (2008). “Identification of diagnostic biomarkers for infection in premature neonates.” Mol Cell Proteomics 7(10): 1863-1875.
29. Kennon, C., G. Overturf, et al. (1996). “Granulocyte colony-stimulating factor as a marker for bacterial infection in neonates.” J Pediatr 128(6): 765-769.
30. Hodge, G., S. Hodge, et al. (2004). “Rapid simultaneous measurement of multiple cytokines using 100 microl sample volumes—association with neonatal sepsis.” Clin Exp Immunol 137(2): 402-407.
31. Martin, H., B. Olander, et al. (2001). “Reactive hyperemia and interleukin 6, interleukin 8, and tumor necrosis factor-alpha in the diagnosis of early-onset neonatal sepsis.” Pediatrics 108(4): E61
32. Santana Reyes, C., F. Garcia-Munoz, et al. (2003). “Role of cytokines (interleukin-1beta, 6, 8, tumour necrosis factor-alpha, and soluble receptor of interleukin-2) and C-reactive protein in the diagnosis of neonatal sepsis.” Acta Paediatr 92(2): 221-227.
33. Schelonka, R. L., A. Maheshwari, et al. (2011). “T cell cytokines and the risk of blood stream infection in extremely low birth weight infants.” Cytokine 53(2): 249-255.
34. Horisberger, T., S. Harbarth, et al. (2004). “G-CSF and IL-8 for early diagnosis of sepsis in neonates and critically ill children—safety and cost effectiveness of a new laboratory prediction model: study protocol of a randomized controlled trial [ISRCTN91123847].” Crit Care 8(6): R443-450.
35. Fischer, J. E., A. Benn, et al. (2002). “Diagnostic accuracy of G-CSF, IL-8, and IL-1ra in critically ill children with suspected infection.” Intensive Care Med 28(9): 1324-1331.
36. Edgar, J. D., V. Gabriel, et al. (2010). “A prospective study of the sensitivity, specificity and diagnostic performance of soluble intercellular adhesion molecule 1, highly sensitive C-reactive protein, soluble E-selectin and serum amyloid A in the diagnosis of neonatal infection.” BMC Pediatr 10: 22.
37. Bernardin G, Strosberg A D, Bernard A, Maffei M, Marullo S. Beta-adrenergic receptor-dependent and -independent stimulation of adenylate cyclase is impaired during severe sepsis in humans. Intensive Care Medicine 1998, 24, 1315-1322.
38. Cao H, Griffin M P, Lake D E, Moorman J R. Increased non-stationarity of neonatal heart rate prior to sepsis and systemic inflammatory response syndrome. Annals of Biomedical Engineering 2004, 32, 233-244.
39. Dellinger R P, Levy M M, Carlet J M, Bion J, Parker M M, Jaeschke R, Reinhart K, Angus D C, Brun-Buisson C, Beale R, Calandra T, Dhainaut J F, Gerlach H, Harvey M, Marini J J, Marshall J, Ranieri M, Ramsay G, Sevransky J, Thompson B T, Townsend S, Vender J S, Zimmerman J L, Vincent J L. Surviving Sepsis Campaign: international guidelines for management of severe sepsis and septic shock: 2008. Crit Care Med. 2008, 36, 296-327.
40. Godin P J, Fleisher L A, Eidsath A, Vandivier R W, Preas H L, Banks S M, Buchman T G, Suffredini A F. Experimental human endotoxemia increases cardiac regularity: results from a prospective, randomized, crossover trial. Crit. Care Med. 1996, 24, 1117-1124.
41. Griffin M P, Lake D, Moorman J R. Heart rate characteristics and clinical signs in neonatal sepsis. Pediatr Res 2007, 61, 222-227.
42. Griffin M P, Lake D E, Bissonette E A, Harrell F E, Jr., O'Shea T M, Moorman J R. Heart rate characteristics: novel physiomarkers to predict neonatal infection and death. Pediatrics 2005, 116, 1070-1074.
43. Griffin M P, Lake D E, Moorman J R. Heart rate characteristics and laboratory tests in neonatal sepsis. Pediatrics 2005, 115, 937-941.
44. Griffin M P, Moorman J R. Toward the early diagnosis of neonatal sepsis and sepsis-like illness using novel heart rate analysis. Pediatrics 2001, 107, 97-104.
45. Griffin M P, O'Shea T M, Bissonette E A, Harrell F E, Jr., Lake D E, Moorman J R. Abnormal heart rate characteristics preceding neonatal sepsis and sepsis-like illness. Pediatr. Res. 2003, 53, 920-926.
46. Griffin M P, O'Shea T M, Bissonette E A, Harrell F E, Jr., Lake D E, Moorman J R. Abnormal heart rate characteristics are associated with neonatal mortality. Pediatr Res 2004, 55, 782-788.
47. Griffin M P, Scollan D F, Moorman J R. The dynamic range of neonatal heart rate variability. J. Cardiovasc. Electrophysiol. 1994, 5, 112-124.
48. Hahn P Y, Yoo P, Ba Z F, Chaudry I H, Wang P. Upregulation of Kupffer cell beta-adrenoceptors and cAMP levels during the late stage of sepsis. Biochimica et Biophysica Acta 1998, 1404, 377-384.
49. Kovatchev B P, Farhy L S, Cao H, Griffin M P, Lake D E, Moorman J R. Sample asymmetry analysis of heart rate characteristics with application to neonatal sepsis and systemic inflammatory response syndrome. Pediatr. Res. 2003, 54, 892-898.
50. Lam H S, Ng P C. Biochemical markers of neonatal sepsis. Pathology. 2008, 40, 141-148.
51. Moorman J R, Lake D E, Griffin M P. Heart rate characteristics monitoring in neonatal sepsis. IEEE Transactions in Biomedical Engineering 2006, 53, 126-132.
52. Neal P R, Kleiman M B, Reynolds J K, Allen S D, Lemons J A, Yu P L. Volume of blood submitted for culture from neonates. J Clin Microbiol. 1986, 24, 353-356.
53. Nelson J C, Rizwan-uddin, Griffin M P, Moorman J R. Probing the order within neonatal heart rate variability. Pediatr Res 1998, 43, 823-831.
54. Oddis C V, Finkel M S. Cytokines and nitric oxide synthase inhibitor as mediators of adrenergic refractoriness in cardiac myocytes. Eur J Pharmacol 1997, 320, 167-174.
55. Oddis C V, Simmons R L, Hattler B G, Finkel M S. Chronotropic effects of cytokines and the nitric oxide synthase inhibitor, L-NMMA, on cardiac myocytes. Biochem Biophys Res Commun 1994, 205, 992-997.
56. Stoll B J, Hansen N, Fanaroff A A, Wright L L, Carlo W A, Ehrenkranz R A, Lemons J A, Donovan E F, Stark A R, Tyson J E, Oh W, Bauer C R, Korones S B, Shankaran S, Laptook A R, Stevenson D K, Papile L A, Poole W K. Late-onset sepsis in very low birth weight neonates: the experience of the NICHD Neonatal Research Network. Pediatrics 2002, 110, 285-291.
57. Tang C, Yang J, Liu M S. Progressive internalization of beta-adrenoceptors in the rat liver during different phases of sepsis. Biochimica et Biophysica Acta 1998, 1407, 225-233.

Unless clearly specified to the contrary, there is no requirement for any particular described or illustrated activity or element, any particular sequence or such activities, any particular size, speed, material, duration, contour, dimension or frequency, or any particularly interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. It should be appreciated that aspects of the present invention may have a variety of sizes, contours, shapes, compositions and materials as desired or required.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims, including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

1. A method of treating an infant suffering from septicemia with an antibiotic, wherein said method comprises:
measuring levels of one or more biochemical substances in at least one sample from an infant;
assessing levels of said one or more biochemical substances against a target value;
interpreting said assessment to provide a determination of a presence of a specific class of infective organism or blood culture result in the sample; and
determining an effective dose of the antibiotic to treat the infant.

2. The method of claim 1, wherein said assessment comprises:
counting the number of said one or more biochemical substances whose levels are above or below a threshold value.

3. The method of claim 1, wherein:
at least one of said one or more samples is a blood sample.

4. The method of claim 2, wherein:
said one or more biochemical substances comprises one or more cytokines.

5-6. (canceled)

7. The method of claim 4, wherein said one or more cytokines comprise at least one of the following:
IL-6;
IL-8;
TNF-α; or
G-CSF.

8. The method of claim 1, wherein said class of infective organism or blood culture result comprises at least one of the following:
Gram-negative;
Gram-positive;
Coagulase-negative staphylococci;
fungus;
virus; or
no growth.

9. The method of claim 7, wherein:
said class of infective organism is Gram-negative; and
said threshold value is about 400 pg/ml for IL-6, about 200 pg/ml for IL-8, about 1000 pg/ml for G-CSF, and about 32 pg/ml for TNF-α.

10. The method of claim 7, wherein:
said class of infective organism is Gram-negative; and
said target value is about 400 pg/ml for IL-6, about 200 pg/ml for IL-8, about 1000 pg/ml for G-CSF, and about 32 pg/ml for TNF-α.

11. (canceled)

12. The method of claim 7, wherein:
said blood culture result is no growth; and
said target value is about 130 pg/ml for IL-6.

13. The method of claim 1, wherein said interpreting comprises:
assigning a score based on said levels such that a higher score indicates a higher probability of the presence of said specific class of infective organism or blood culture result.

14-15. (canceled)

16. The method of claim 1, further comprising:
measuring heart rate characteristics or other physiologic measures; and
wherein said interpreting incorporates analysis of said heart rate characteristics or other physiologic measures.

17-32. (canceled)

33. A non-transitory computer useable medium having a computer program logic for treating an infant suffering from septicemia with an antibiotic, said computer program logic causing at least one processor to:
measure levels of one or more biochemical substances in at least one sample from an infant;
identify and counting the number of said biochemical substances whose levels are above a threshold value; and
interpret said measures of said one or biochemical substances to provide a determination of a presence of a specific class of infective organism or blood culture result in the sample; and
determine an effective dose of the antibiotic to treat the infant.

34. The non-transitory computer useable medium of claim 33, wherein said determining comprises:
counting the number of said one or more biochemical substances whose levels are above or below a threshold value.

35. The non-transitory computer useable medium of claim 33, wherein:
said sample is a blood sample.

36. The non-transitory computer useable medium of claim 33, wherein:
said one or more biochemical substances comprises one or more cytokines.

37-38. (canceled)

39. The non-transitory computer useable medium of claim 36, wherein said one or more cytokines comprise at least one of the following:
IL-6;
IL-8;
TNF-α; or
G-CSF.

40. The non-transitory computer useable medium of claim 33, wherein said class of infective organism or blood culture result comprises at least one of the following:
gram-negative;
gram-positive;
coagulase-negative staphylococci;
fungus;
virus; or
no growth.

41-42. (canceled)

43. The non-transitory computer useable medium of claim 33, further comprising:
measuring heart rate characteristics or other physiologic measures; and
wherein said interpreting incorporates analysis of said heart rate characteristics or other physiologic measures.
